(19) United States
(12) Patent Application Publication
(10) Pub. No.: US 2026/0242718 A1
(43) Pub. Date: Aug. 20, 2026

(54) ELECTRODE-INTEGRATED MICROWELL ARRAY SYSTEM FOR MULTI-MODAL ELECTRICAL MONITORING OF THREE-DIMENSIONAL BIOLOGICAL SAMPLES

(71) Applicant: University of Denver, Denver, CO (US)

(72) Inventors: Seyed Reza Mahmoodi, Denver, CO (US); Derick Irawan, Denver, CO (US); Bryce Clinkenbeard, Denison, IA (US); Mariana Neuenschwander Martins, Denver, CO (US)

(21) Appl. No.: 19/544,690

(22) Filed: Feb. 19, 2026

Related U.S. Application Data
(60) Provisional application No. 63/761,072, filed on Feb. 20, 2025.

(51) Int. Cl. C12M 1/34; C12M 1/32; C12M 3/06; G01N 27/02; G01N 33/483

(52) U.S. Cl. CPC C12M 41/46; C12M 23/12; C12M 23/16; G01N 27/02; G01N 33/4833

(57) ABSTRACT

A system and method for multi-modal monitoring of three-dimensional cell cultures that includes a platform including a plurality of microwells, each microwell of the plurality of microwells being defined by a sidewall and a bottom surface. The system and method further include a first electrode structure disposed within each microwell, the first electrode structure comprising a pair of coplanar conductive elements configured to obtain a two-dimensional impedance measurement, and a second electrode configured to cooperate with at least a portion of the first electrode structure to obtain a three-dimensional impedance measurement using at least one conductive element of the first electrode structure and the second electrode.

### CROSS-REFERENCE TO RELATED APPLICATIONS

This application claims priority to U.S. application Ser. No. 63/761,072, titled Electrode integrated array platform for non-invasive, multi-modal monitoring of 3D cell cultures, filed Feb. 20, 2025, which is hereby incorporated by reference in its entirety.

### SPONSORED RESEARCH AND DEVELOPMENT

This invention was made with government support under Grant No. CTGG1 2025-3952 awarded by the Colorado Office of Economic Development and International Trade (OEDIT) Advanced Industries Proof of Concept Grant. The government has certain rights in the invention.

### FIELD OF DISCLOSURE

The present disclosure relates to sensor systems for monitoring biological samples, and more particularly to an electrode-integrated microwell array platform for non-invasive, multi-modal, real-time monitoring of three-dimensional biological samples.

### BACKGROUND

Three-dimensional cell structures, also referred to as microphysiological systems (MPSs), provide valuable alternatives to animal testing for studying disease pathogenesis and cellular pathology. These 3D cell structures can originate from various sources, including primary tissue, stem cells, and established cell lines. A combination of 3D cultures and microfluidic devices can enhance these models by closely replicating in-vivo conditions, improving the study of microphysiological systems and cellular interactions.

Electrical sensing offers a non-invasive and real-time alternative for monitoring cells in both 2D and 3D cultures. These methods can readily be applied in an incubator, making them suitable for integration into automated systems for multiplexed probing. Optical imaging techniques such as continuous microscopy, time-lapse microscopy, and luminescence-or fluorescence-based assays have been used for monitoring 3D cultures.

### BRIEF DESCRIPTION OF FIGURES

Non-limiting and non-exhaustive examples are described with reference to the following figures.

FIG. 1A shows a longitudinal cross section of an illustrative microchannel depicting a two-dimensional mode of impedance data collection, in accordance with various embodiments disclosed herein.

FIG. 1B shows a transverse cross section of an illustrative microchannel depicting a three-dimensional mode of impedance data collection, in accordance with various embodiments disclosed herein.

FIG. 2A shows an illustrative conventional electric cell-substrate impedance sensing configuration with a spheroid positioned above an electrode, in accordance with various embodiments disclosed herein.

FIG. 2B shows an illustrative electrode configuration with a top electrode and a bottom electrode for enhanced electric field distribution through a spheroid, in accordance with various embodiments disclosed herein.

FIG. 2C shows an illustrative electrode configuration with a sample positioned between a top electrode and a bottom electrode, in accordance with various embodiments disclosed herein.

FIG. 2D shows an illustrative configuration for inducing cell aggregation and spheroid formation using electrical forces and fluid flow, in accordance with various embodiments disclosed herein.

FIG. 3A shows an illustrative microfabrication process for building concave microwell arrays with stacked sensing electrodes, in accordance with various embodiments disclosed herein.

FIG. 3B shows an illustrative microfluidic chip design with a microwell array and serpentine channels, in accordance with various embodiments disclosed herein.

FIG. 4A shows an illustrative cross-sectional view of a microwell with electrodes showing electric field distribution at different frequencies, in accordance with various embodiments disclosed herein.

FIG. 4B shows an illustrative graph of impedance changes over time during spheroid development, in accordance with various embodiments disclosed herein.

FIG. 4C shows an illustrative graph of impedance versus frequency for treatment and control groups, in accordance with various embodiments disclosed herein.

FIG. 5 shows an illustrative multi-modal sensing system for monitoring and characterizing three-dimensional cell cultures, in accordance with various embodiments disclosed herein.

FIG. 6A shows an illustrative cross-sectional view of a microfluidic channel configuration showing electric field distribution, in accordance with various embodiments disclosed herein.

FIG. 6B shows an illustrative multiplexed device integrated with cytometers for spheroid formation control, in accordance with various embodiments disclosed herein.

FIG. 7 shows an illustrative automated microfluidic platform for label-free quantitative assessment of spheroid viability, in accordance with various embodiments disclosed herein.

FIG. 8 shows an illustrative isometric view of a microwell array with a fluidic containment block, in accordance with various embodiments disclosed herein.

FIG. 9 shows an illustrative isometric view of an arrayed alternative electrode configuration for a microwell array platform, in accordance with various embodiments disclosed herein.

FIG. 10A shows an illustrative isometric view of an alternative electrode configuration for a microwell, in accordance with various embodiments disclosed herein.

FIG. 10B shows an illustrative cross-sectional side view of a microwell configuration with electrodes, in accordance with various embodiments disclosed herein.

FIG. 10C shows an illustrative top view of a microwell configuration with electrode arrangement, in accordance with various embodiments disclosed herein.

FIG. 11A shows an illustrative top view of a collective continuous electrode configuration for a microwell array, in accordance with various embodiments disclosed herein.

FIG. 11B shows an illustrative photographic image of an arrayed electrode configuration with multiple microwells, in accordance with various embodiments disclosed herein.

FIG. 12 shows an illustrative impedance Bode plot showing electrical properties of a microwell array during spheroid formation over time, in accordance with various embodiments disclosed herein.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

These and other features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a,’ ‘an,’ and ‘the’ include plural referents unless the context clearly dictates otherwise.

In the description herein, various ranges and/or numerical limitations can be expressly stated below. It should be recognized that unless stated otherwise, it is intended that endpoints are to be interchangeable. Further, any ranges include iterative ranges of like magnitude falling within the expressly stated ranges or limitations.

Furthermore, various modifications can be made within the scope of the invention as herein intended, and embodiments of the invention can include combinations of features other than those expressly claimed. In particular, flow arrangements other than those expressly described herein are within the scope of the invention.

Regarding claim transitional terms or phrases, the transitional term “comprising”, which is synonymous with “including,” “containing,” “having” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter composition or method to which the term is applied.

Within this specification, use of “comprising” or an equivalent expression contemplates the use of the phrase “consisting essentially of,” “consists essentially of,” or equivalent expressions as alternative embodiments to the open-ended expression. Additionally, use of “comprising” or an equivalent expression or use of “consisting essentially of” in the specification contemplates the use of the phrase “consisting of,” “consists of,” or equivalent expressions as an alternative to the open-ended expression or middle ground expression, respectively. For example, “comprising” should be understood to include “consisting essentially of,” and “consisting of” as alternative embodiments for the aspect, features, and/or elements presented in the specification unless specifically indicated otherwise.

While compositions and methods are described in terms of “comprising” various components or steps, the compositions and methods can also “consist essentially of” or “consist of” the various components or steps.

For purposes of this disclosure, the term “three-dimensional (3D) cell culture” refers broadly to a cellular assembly in which cells are organized in a three-dimensional architecture that more closely mimics in vivo tissue structure than monolayer two-dimensional cultures. A 3D cell culture may include, without limitation, spheroids, organoids, microtissues, microphysiological systems (MPSs), organ-on-chip constructs, vascularized tissue constructs, tumor aggregates, neurospheres, embryoid bodies, stem cell aggregates, or combinations thereof. Unless otherwise specified, the term “3D cell culture” is intended to encompass both scaffold-free and scaffold-based cellular assemblies.

As used herein, the term “spheroid” refers to a generally rounded or ellipsoidal aggregate of cells formed through cell-cell adhesion in the absence of an external supporting scaffold or within a minimally restrictive environment. Spheroids may be formed from a single cell type or multiple cell types and may include tumor spheroids, epithelial spheroids, stem cell-derived spheroids, or co-culture spheroids.

As used herein, the term “organoid” refers to a self-organizing three-dimensional cellular structure derived from stem cells, progenitor cells, or primary tissue that recapitulates at least one structural or functional aspect of a native organ. Organoids may exhibit spatial organization, differentiation patterns, or physiological responses representative of the corresponding in vivo tissue.

The term “microphysiological system” (MPS) refers to an engineered in vitro tissue model that integrates cellular constructs with microfluidic, mechanical, electrical, or biochemical components to simulate aspects of tissue-level physiology. MPS platforms may include vascularized microtissues, barrier models, or organ-on-chip devices incorporating perfusion and environmental control.

The term “sample,” as used throughout this disclosure, refers to any 3D cell culture, cell aggregate, microtissue, organoid, or biological construct disposed within a microwell or fluidic region of the platform. The term “sample” is used as a general reference to avoid limiting embodiments to a particular tissue type. Accordingly, references in the figures to “vascular tissue” are illustrative examples of one possible sample type and are not intended to limit the scope of the disclosure unless explicitly stated. In various embodiments, the sample may comprise epithelial tissue, vascular tissue, neuronal tissue, tumor tissue, stem cell aggregates, or other multicellular constructs.

Unless otherwise specified, references to “cell culture,” “3D culture,” “microtissue,” “vascular tissue,” “spheroid,” or “organoid” in the description and figures may be used interchangeably to describe representative implementations of a sample within the microwell array platform. The systems and methods described herein are not limited to a specific tissue class and may be applied to a broad range of three-dimensional cellular assemblies.

Three-dimensional cell structures originate from various sources, including primary tissue, stem cells (SCs), and established cell lines. These structures may be sensitive to small variations such as cell density, procedure, number of passages, surface coating, and medium composition, resulting in differences in properties, stage of differentiation, and cell types. For instance, microphysiological systems (MPSs) based on induced pluripotent stem cells (iPSCs) require careful monitoring and optimization of culture conditions. The challenge arises from the heterogeneity of cell cultures, leading to difficulties in reproducing data within the same study or across different studies. This concern becomes more pronounced in the context of 3D cultures, where the complexity of the cellular environment can introduce additional variables that affect experimental reproducibility.

Although efforts have been made to promote in vitro microfluidic models for simulating physiological environments in a well-defined and reproducible manner, these devices often rely on microscopy techniques involving labels, off-chip measurements, or the need to terminate the experiment for various cellular or molecular analyses. Additionally, continuous microscopy of microfluidic-based systems faces several limitations, including limited imaging depth and spatial resolution, light scattering, and stability issues. These challenges become particularly problematic for real-time monitoring of 3D cell structures, such as spheroids and organoids, with typical diameters of at least several hundred micrometers. Moreover, optical imaging techniques are not easily scalable for parallel in-situ analysis, and high-throughput screening and discoveries remain a challenge. Consequently, time-lapse microscopy and cellular metabolic activity assays based on luminescence or fluorescence are considered the current state of the art for analyzing drug responses in 3D cultures, despite their limitations in enabling real-time and efficient recording of drug action.

Alternatively, electrical sensing offers a non-invasive and real-time solution for monitoring cells in both 2D and 3D cultures, providing advantages in experimental setups. These methods can readily be applied in an incubator, making them suitable for integration into automated systems for multiplexed probing. The technical challenge for electrical monitoring of 3D models lies in achieving a sufficient and uniform electric field throughout the cell culture in all directions for high spatiotemporal resolution and high-content data collection. Bridging this challenge can be beneficial for the label-free, quantitative, and continuous assessment of cell viability parameters and medium properties, representing a technical challenge in advancing in vitro 3D models and Organ-on-chip (OoC) systems.

A reconfigurable multisensor-integrated microfluidic platform capable of producing viable, same-sized 2D or 3D cell structures may provide a versatile foundation for comprehensive and dynamic analysis of cellular behavior. Such a system may minimize potential interference or artifacts caused by labeling and human error, saving time and costs associated with treatments, making the system a valuable tool for various applications in precision medicine, drug discovery, and tissue engineering, and a component for achieving the goals of OoC technology.

The present disclosure describes various embodiments of a sensor system and a sensor method including an electrode-integrated microwell array platform capable of capturing high-content, multi-modal data in a non-invasive manner with highly sensitive spatiotemporal resolution, while being fully compatible with standard laboratory equipment and incubators. Various embodiments described herein achieve high spatiotemporal resolution, which allows for non-invasive, real-time, and precise impedance monitoring of 3D cultures.

In various embodiments, by incorporating electrodes into the microwell array, the platform can advantageously overcome challenges in cell and tissue engineering, particularly the inherent limitations of traditional microscopy and optical imaging techniques used to monitor 3D cell models. In some aspects, the platform's electrical data can advantageously offer a more direct, quantifiable, and consistent output compared to image analysis, reducing variability and enhancing interpretability. Furthermore, this streamlined data format may be suited for integration with artificial intelligence (AI), facilitating the training of predictive models for more accurate and reliable analysis.

In some aspects, microfabrication techniques may be used to vertically stack microelectrodes around and within microwells disposed on a platform. In various embodiments, this configuration boosts the spatial resolution over existing monitoring solutions, thus addressing the challenge of achieving continuous, non-invasive monitoring in electrical assessments of 3D models. Achieving high resolution signal enables in-situ monitoring of gradual inter-and intra-cellular activities, without the need for expensive instrumentation or interrupting the experiment for labeling and off-plate measurements. Further, stacked microelectrodes can be operated as a 2D array, to obtain electrical measurements in a plane across a top or bottom of the platform, as well as in a 3D configuration, to obtain electrical measurements across each microwell, thus enabling multi-modal monitoring of 3D cell cultures. Various embodiments described herein help minimize interference, off-plate measurement artifacts, labeling, and human error, enhancing standardization.

In some embodiments, optionally, the use of transparent electrodes (such as indium-tin-oxide (ITO) patterned electrodes) enable time-lapse imaging of spheroids, which can be beneficial for benchmarking and calibrating the spheroid viability assay alongside optical monitoring. For example, ITO electrodes may be used when optical monitoring is desired alongside electrical sensing. In some embodiments, non-transparent electrodes, such as those formed with gold or platinum, can be advantageous in providing superior conductivity without optical interference.

Multiple advantages are provided by the various embodiments disclosed herein. For example, various embodiments described herein enable the capture of high-resolution, high content electrical data through multimodal monitoring. This type of data can be particularly advantageous since electrical signal recordings may be more easily integrated with post processing, such as with machine learning algorithms for predictive data modeling, compared to more complex data sets such as microscopy images. As a result, various embodiments described herein may achieve improved or high resolution signals and improved or optimized multimodal electrical data integration, leading to better data interpretation and predictive accuracy.

In some embodiments, the system features a platform with one or more sample wells including two sets of counter electrodes that enable monitoring of the samples within the wells in alternating 2D (in-plane) and 3D (out-of-plane) configurations. The 2D and 3D configurations can even be used to monitor the 3D culture simultaneously in planar and volumetric manners. That is, in various embodiments described herein simultaneous characterization of spheroids is enabled using two different electrode schemes to discriminate between bulk spheroid interactions (cell-to-cell dynamics) and the spheroid surface properties (cell-to-media interactions). Such sensitive multimodal monitoring can advantageously deliver insights into, for example, gradual cellular responses and drug efficacy, thus enhancing accuracy and adaptability for various disease models.

In some embodiments, the sample may comprise an epithelial spheroid formed from epithelial cell lines or primary epithelial cells. For example, epithelial cells may be introduced into the plurality of microwells under static or perfused conditions and allowed to aggregate to form spheroids through sedimentation and cell-cell adhesion. In such embodiments, the two-dimensional impedance measurement mode may be particularly sensitive to cell-to-media and cell-to-extracellular matrix (ECM) interactions occurring at the outer surface of the spheroid. The three-dimensional impedance measurement mode may enable detection of changes in epithelial barrier integrity, tight junction formation, proliferation, drug-induced cytotoxicity, or necrotic core development within the spheroid. Frequency-dependent impedance measurements may distinguish surface barrier properties from bulk cytoplasmic conductivity changes during spheroid maturation or treatment response.

In various embodiments, the sample may comprise a vascular microtissue, endothelial microtissue, or perfusable vascularized construct. For example, endothelial cells may be cultured within concave microwells under controlled perfusion to form vascular spheroids or microvascular assemblies. In such embodiments, the three-dimensional impedance measurement mode may enable volumetric assessment of cell-cell junction integrity, angiogenic sprouting behavior, and microvascular remodeling. The platform may additionally be configured to measure trans-endothelial electrical resistance (TEER) using the stacked electrode configuration without requiring a conventional Transwell system. In some embodiments, this measurement may correspond to transepithelial or transendothelial electrical impedance representative of barrier integrity in three-dimensional constructs. Variable fluid shear stress applied through the microfluidic channels may be used to simulate physiological or pathological flow conditions while impedance monitoring captures dynamic changes in vascular barrier function.

In further embodiments, the sample may comprise neuronal spheroids, tumor organoids, stem cell-derived aggregates, co-culture constructs, or disease-specific microtissues. The multimodal impedance measurements described herein may enable differentiation of electrophysiological activity, proliferation dynamics, hypoxic core formation, and therapeutic response across these distinct tissue classes.

In various embodiments, a system for multi-modal monitoring of three-dimensional biological samples includes a platform having a plurality of microwells, each microwell defined by a sidewall and a bottom surface and configured to receive a sample. A first electrode structure is disposed within each microwell. The first electrode structure includes a pair of coplanar conductive elements positioned relative to one another along a common plane at or near the bottom surface of the microwell. A second electrode is disposed adjacent to each microwell and positioned relative to the first electrode structure such that an electric field may be established across at least a portion of a depth of the microwell.

The pair of coplanar conductive elements of the first electrode structure are independently controllable to obtain a two-dimensional impedance measurement. In the two-dimensional mode, an electrical signal is applied between the pair of coplanar conductive elements such that current flows primarily along a planar path near the bottom surface of the microwell. This planar measurement may be sensitive to surface-level interactions between the sample and surrounding medium.

At least a portion of the first electrode structure and the second electrode are controllable to obtain a three-dimensional impedance measurement. In the three-dimensional mode, an electrical signal is applied between at least one conductive element of the first electrode structure and the second electrode such that an electric field extends across a depth of the microwell. The resulting impedance measurement may be sensitive to bulk or volumetric properties of the sample disposed within the microwell.

In some embodiments, the first electrode structure includes a spiral-shaped coplanar electrode pair having two electrically isolated conductive traces arranged in a coiled or interleaved geometry. The conductive traces may function as a sending path and a receiving path, or as working and counter electrodes, such that planar impedance measurements are performed without requiring activation of the second electrode.

In various embodiments, a spacing between the first electrode structure and the second electrode is selected based on an expected dimension of a three-dimensional biological sample disposed within the microwell. The spacing may be in a range of about 300 microns to about 500 microns to enable through-depth impedance measurements across typical spheroid dimensions.

In some embodiments, the system further includes a microfluidic circuit in fluid communication with the plurality of microwells. The microfluidic circuit may include interconnected microchannels, serpentine channels, distribution manifolds, inlet ports, and outlet ports configured to deliver and remove fluid relative to the microwells. The microfluidic circuit may operate under static, perfused, recirculating, or dynamically controlled flow conditions.

In various embodiments, a method for monitoring three-dimensional cell cultures includes providing a platform having a plurality of microwells, introducing cells into the microwells, applying an electrical signal between at least one conductive element of a first electrode structure and a second electrode to obtain a three-dimensional impedance measurement, and monitoring formation or viability of a cell culture based on the impedance measurement. The method may further include applying an electrical signal between the pair of coplanar conductive elements of the first electrode structure to obtain a two-dimensional impedance measurement.

In some embodiments, application of an electrical field between the first electrode structure and the second electrode induces aggregation of cells into a spheroid within the microwell. Adjustment of magnitude and frequency of the electrical field may control spheroid size and formation dynamics.

In various embodiments, a microfluidic device for monitoring three-dimensional cell cultures includes a microwell array having a plurality of microwells configured to accommodate spheroid formation, a first electrode layer disposed within the microwell array, and a second electrode layer disposed at a top position of the microwell array. In some embodiments, the first electrode layer includes a plurality of electrode structures, and each electrode structure can include a pair of coplanar conductive elements positioned within a corresponding microwell. In some aspects, the second electrode layer is positioned adjacent to the plurality of microwells such that impedance measurements may be performed across a depth of each microwell.

In some embodiments, at least one of the first electrode layer or the second electrode layer includes a transparent conductive material, including indium-tin-oxide, enabling simultaneous optical imaging and electrical monitoring. In other embodiments, the electrode layers include conductive metals such as gold or platinum to provide enhanced conductivity for electrical measurements.

In some embodiments, the system further includes an analog multiplexer configured to selectively connect to individual microwells of the plurality of microwells. A microprocessor may generate control signals for the analog multiplexer to switch between the two-dimensional impedance measurement and the three-dimensional impedance measurement, thereby enabling selective activation of electrode pairs without manual reconnection.

In various embodiments, the first electrode layer enables planar impedance measurements within a bottom region of each microwell by applying an electrical signal between the pair of coplanar conductive elements positioned within the corresponding microwell.

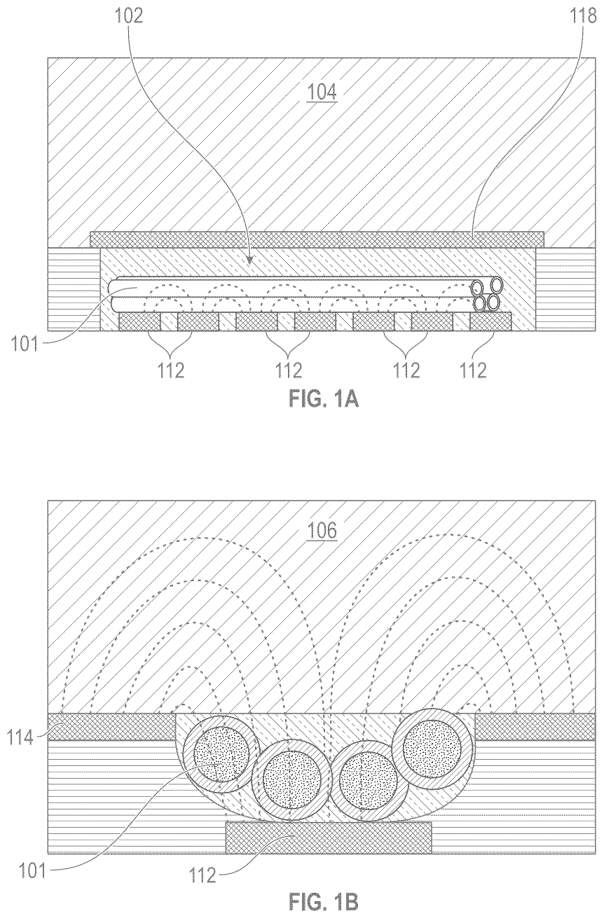

Referring to FIGS. 1A and 1B, longitudinal and transverse cross sections of microchannels are shown, demonstrating two distinct modes of impedance data collection in accordance with various embodiments. FIG. 1A illustrates a longitudinal cross section depicting a 2D mode 104 of impedance data collection. The configuration includes a microfluidic channel cover 118 positioned above a microwell 102. In some aspects, a sample 101 (e.g., a vascular microtissue, epithelial spheroid, organoid, or other 3D cell culture) may be disposed within the microwell 102 and a spiral electrode structure 112 comprising a pair of coplanar conductive traces may be positioned at a bottom surface of the microwell 102, beneath the sample 101. In the 2D mode 104, the spiral electrode structure 112 may enable planar impedance measurements from a surface of the sample 101, capturing data related to cell-to-media interactions at an interface between the sample 101 and surrounding medium. In the 2D mode 104, current flows between the two coplanar conductive traces of the spiral electrode structure 112, and the top electrode 114 is not required for planar impedance sensing.

In various embodiments, element 112 (referred to herein as the “spiral electrode structure”) comprises a coplanar electrode structure including at least two electrically isolated conductive elements arranged in a spiral or coiled geometry. The conductive elements may function as a sending electrode and a receiving electrode, or as working and counter electrodes, configured to pass current between the conductive elements along a planar path. Accordingly, references herein to the “spiral electrode structure 112” describe a planar electrode assembly comprising multiple conductive traces rather than a single monolithic conductor.

As used herein, the term “first electrode structure” refers to an electrode assembly disposed within a microwell and comprising at least two electrically isolated conductive elements configured to enable planar impedance measurements. The first electrode structure may include, in certain embodiments, a spiral electrode structure 112 comprising a pair of coplanar conductive elements arranged in a spiral or interleaved geometry. References to the “spiral electrode 112” refer to this embodiment of the first electrode structure and should be understood to encompass the pair of coplanar conductive elements forming the planar sensing assembly.

With continued reference to FIG. 1B, a transverse cross section is shown depicting a 3D mode 106 of impedance data collection. In various aspects, this configuration includes the microfluidic channel cover 118 positioned above the microwell 102, with the sample 101 disposed within the microwell 102. In the 3D mode 106, at least one conductive element of the spiral electrode structure 112 cooperates with the second electrode 114 to establish an electric field extending across a depth of the microwell. In various embodiments, the second electrode 114 may be positioned at a top region of the microwell, integrated into a cover layer, disposed proximate to an opening of the microwell, incorporated into a lid or microfluidic channel layer, or otherwise spatially arranged relative to the spiral electrode structure 112 such that an electric field is established through at least a portion of the depth of the microwell. The second electrode 114 may be continuous, segmented, ring-shaped, planar, or partially surrounding the microwell. The 3D mode 106 may use the top electrode 114 and the spiral electrode structure 112 as counter electrodes, enabling volumetric impedance measurements throughout the sample 101. In various embodiments, the second electrode may be positioned at a top region of the microwell, integrated into a cover layer, disposed along one or more sidewalls of the microwell, or otherwise spatially arranged relative to the first electrode structure such that an electric field is established across at least a portion of a depth of the microwell. As used herein, “sidewall” refers to one or more structural surfaces extending between a bottom surface and an opening of a microwell, and may include straight, tapered, curved, conical, cylindrical, faceted, or otherwise contoured surfaces defining a cavity.

The second electrode need not be directly aligned with a center of the microwell and may include segmented, ring-shaped, or partially surrounding geometries configured to shape the electric field distribution. For example, in some aspects, this electrode arrangement may establish an electric field distribution across an entire depth of the microwell 102, providing improved or high spatiotemporal resolution for characterizing bulk tissue properties and cell-to-cell interactions within the three-dimensional structure.

In some embodiments, the electrodes described herein may be in direct conductive contact with the culture medium, partially insulated from the medium, capacitively coupled through a dielectric layer, or otherwise configured to establish an electric field within the microwell without requiring direct electrical conduction between the electrode surface and the sample. Accordingly, impedance or electrical measurements may be performed using conductive, capacitive, or field-coupled configurations, provided that an electric field interaction with the sample is established for planar and/or through-depth characterization.

In various embodiments, the dual monitoring electrode design enables simultaneous acquisition of high-resolution, multi-modal data from embedded microchannel arrays, allowing differentiation between surface dynamics captured in the 2D mode 104 and bulk cellular properties captured in the 3D mode 106. The spiral electrode structure 112 may be configured as a coplanar interdigitated or double-spiral electrode pair, wherein current is passed between the two coplanar conductive elements to obtain planar (2D) impedance measurements sensitive to surface-level events. In various embodiments, the spiral electrode structure 112 comprises a co-planar electrode pair including two electrically isolated conductive traces (e.g., a sending path and a receiving path) arranged in a spiral or double-spiral configuration. The two conductive traces may be twisted, interleaved, or coiled around one another in a manner analogous to comb-like interdigitated electrodes (IDEs), such that current may be passed between the traces to perform planar (2D) impedance measurements without requiring the top electrode 114. Because the conductive traces are separated by a small in-plane gap, the resulting electric field is primarily confined near the substrate, advantageously making the 2D mode highly sensitive to surface interactions between the sample 101 and the surrounding medium. A planar 2D electrode spacing may be configured to be between 10 and 100 microns to ensure sensitivity for measuring surface events at the interface between the sample 101 and the surrounding medium.

In various embodiments, in contrast to conventional rectangular interdigitated electrodes, the spiral geometry provides a radially distributed sensing region that aligns with the typical circular growth pattern of spheroids. This configuration advantageously promotes more uniform sensitivity across a base of the three-dimensional structure and reduces directional bias that may arise from linear electrode arrangements. In some embodiments, the electrodes may be formed of a transparent conductive material such as indium-tin-oxide (ITO) to enable simultaneous optical imaging and electrical measurements. The use of transparent electrodes can allow for time-lapse imaging of samples contained within the microwell 102 while electrical monitoring is performed concurrently. In various aspects, the electrodes can be formed of non-transparent materials such as gold or platinum to provide superior conductivity without optical interference. In some aspects, the selection of electrode material may depend on whether optical monitoring is desired alongside electrical sensing.

In some embodiments, the 3D electrode spacing may be designed to be proportional to a desired spheroid diameter. For example, a microwell height and 3D electrode spacing may be formed in a range of 300 to 500 microns, matching typical spheroid sizes. In some embodiments, the spacing may be about 300 to about 500 microns. Narrower ranges within this range may also be used depending on the spheroid dimensions being monitored, such as about 300 to about 400 microns, about 350 to about 450 microns, about 400 to about 500 microns, or other intermediate values within this range. Such designs may advantageously ensure high spatial sensitivity without introducing excess noise into captured data. In some aspects, the spacing between the top electrode 114 and the spiral electrode structure 112 may be configured to accommodate the dimensions of the sample 101 or spheroid contained within the microwell 102, enabling improved accuracy and sensitivity of impedance measurements across the three-dimensional structure.

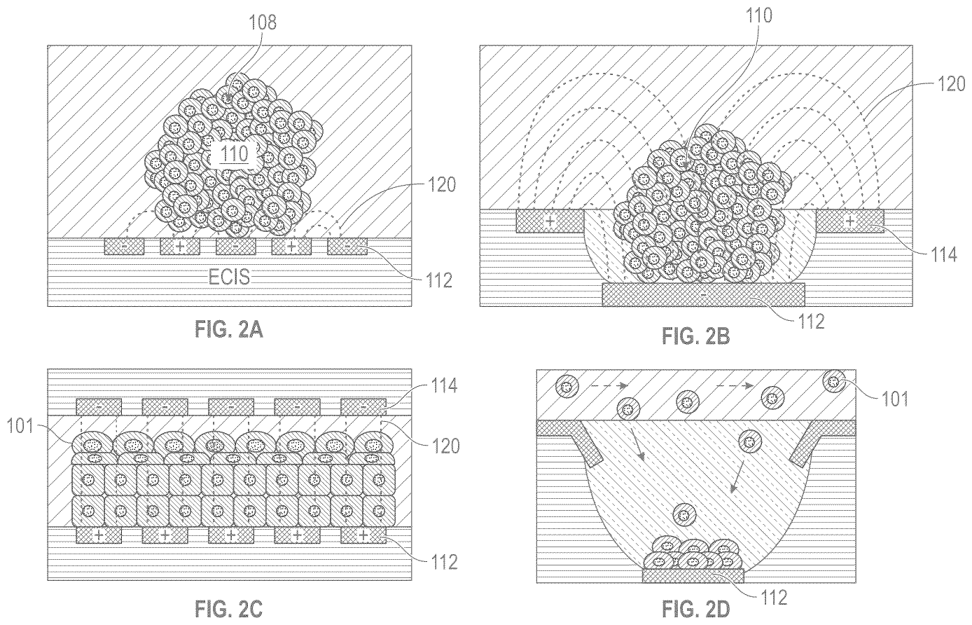

Referring to FIGS. 2A-2D, variations of electromagnetic field formation and their effects on spheroid formation are illustrated in accordance with various embodiments of the present disclosure. FIG. 2A depicts a conventional electric cell-substrate impedance sensing (ECIS) configuration where a 3D spheroid 110 is positioned above the spiral electrode structure 112. In this arrangement, an electric field distribution 120 is concentrated primarily at a bottom surface near the spiral electrode structure 112. The electric field distribution 120 corresponds to current flow paths through the microtissue. This configuration results in blind spots 108 in regions of the 3D spheroid 110 that are located away from the electrode plane. The blind spots 108 represent areas of the 3D spheroid 110 that are difficult to characterize using conventional ECIS techniques due to insufficient electric field penetration into the three-dimensional structure.

With continued reference to FIG. 2B, in some aspects an improved configuration is shown where the top electrode 114 is positioned above the 3D spheroid 110 in addition to the spiral electrode structure 112 at the bottom. This arrangement can advantageously enable the electric field distribution 120 to extend throughout the 3D spheroid 110, providing enhanced spatial resolution and eliminating the blind spots 108 that occur in the conventional ECIS configuration shown in FIG. 2A. The top electrode 114 and the spiral electrode structure 112 may function as counter electrodes to establish voltage across the 3D spheroid 110, enabling spatial input signal recognition throughout the three-dimensional structure.

FIG. 2C illustrates a configuration with the sample 101 positioned between the top electrode 114 and the spiral electrode structure 112. The integration of transparent electrode pairs on both the top and bottom of the channel may improve spatial resolution without obstructing light. In some aspects, by transforming the conventional ECIS planar electrode configuration into a 3D arrangement of fully transparent electrodes, this approach can foster interactions between electrodes and 3D cellular structures while retaining the benefits of fluorescence microscopy and live-cell imaging. The electric field distribution 120 may extend between the electrodes, enabling characterization of the sample 101 in a three-dimensional manner.

As further shown in FIG. 2D, a combination of fluid flow drag and electrical forces can be used to induce cell aggregation and spheroid formation. The electric field distribution 120 generated between the top electrode 114 and the spiral electrode structure 112 can facilitate the movement and assembly of cells within the microwell to achieve uniformly sized spheroids. By adjusting a magnitude and frequency of an applied electrical field and controlling a flow rate, cells can be induced to aggregate and form spheroids with consistent dimensions. In various aspects, this label-free and contactless patterning promotes the formation of spheroids of desired sizes at higher throughput, with accurate size control, higher reproducibility, cell viability, and adaptability to different cell lines.

In some embodiments, the top electrode 114 may be fully coated with a conductive material to function as a continuous electrode. The 2D electrodes, such as the spiral electrode structure 112, may be individually addressed to allow detection of microwell-to-microwell interactions. For example, for electrogenic cells, top layer microwell electrodes may be segmented to pick up cell signaling, which can complement impedance-based trend analyses. In various aspects, the 3D electrodes may be symmetrically arranged around the microwell to provide uniform electric field distribution throughout the 3D spheroid 110 or the sample 101.

In some embodiments, non-uniform or asymmetric electromagnetic fields are established in the 3D mode 106 to provide dielectrophoretic effects for enhanced cell seeding and spheroid formation. By combining electrophoretic and dielectrophoretic forces, the formation of uniformly sized and shaped spheroids is facilitated, enhancing cell seeding efficiency. In various aspects, in these systems, the 3D electrode arrangement may reduce the need for electrochemical impedance spectroscopy (EIS), a process that may have challenging data interpretation, time consumption, and variability in execution. For example, the 3D electrode arrangement may reduce reliance on complex multi-frequency electrochemical impedance spectroscopy (EIS) analysis in certain embodiments by enabling improved spatial resolution and field uniformity, thereby allowing meaningful characterization using reduced frequency sets or simplified impedance measurements. The enhanced spatial resolution provided by the microwell-integrated electrodes can offer valuable insights into a more accurate representation of disease state, spheroid heterogeneity, and monitoring of gradual cellular responses.

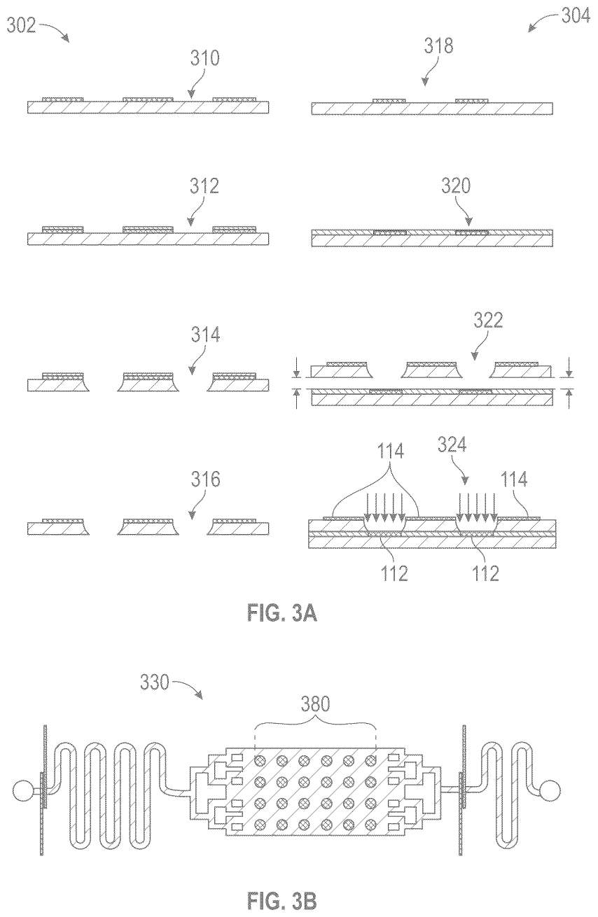

Referring to FIGS. 3A and 3B, microfabrication steps for building concave microwell arrays incorporated with stacked sensing electrodes and a microfluidic biochip design for creating uniform flow are illustrated in accordance with various embodiments. FIG. 3A depicts a series of cross-sectional views showing a fabrication process for both a top layer 302 and a bottom layer 304. In some embodiments, the microfabrication process may include photolithography, soft lithography, and plasma etching to create a microwell array 380 with stacked counter electrodes.

On a left side of FIG. 3A, various embodiments of a fabrication sequence for the top layer 302 is shown. The fabrication sequence begins with patterning ITO electrodes on the top layer 310, which shows a substrate with patterned transparent conductive material. Step 312 involves patterning resist, where a photoresist layer is applied over the patterned electrodes. Following this, isotropic etching of glass 314 creates concave microwell structures in the substrate, resulting in separated well formations. The sequence concludes with resist removal 316, leaving the etched microwell structures with the patterned electrodes.

With continued reference to FIG. 3A, on a right side, various embodiments of a fabrication sequence for the bottom layer 304 is shown. The fabrication sequence for the bottom layer 304 begins with patterning ITO electrodes on the bottom layer 318, showing a substrate with patterned transparent conductive electrodes. Step 320 involves PDMS prepolymer coating, where a layer of polydimethylsiloxane (PDMS) prepolymer is applied over the patterned electrodes. Bonding of wafers (PDMS curing) 322 joins the top layer 302 and the bottom layer 304 together, with the top electrode 114 positioned at an upper surface and the spiral electrode structure 112 positioned at a lower surface. A final step involves oxygen plasma etching 324, which is applied to create the completed microwell structure with integrated electrodes on both surfaces.

In some aspects, the microfabrication process stacks the two counter electrodes into the microwells to create the microwell array 380. ITO electrodes are highly transparent, which allows for non-invasive live imaging of cells and tissues using various optical microscopy techniques. The transparency of the ITO electrodes can enable time-lapse imaging of spheroids for benchmarking against impedance data in both the 2D mode 104 and the 3D mode 106, and may facilitate assay self-calibration. In various aspects, the platform may use SU-8 micropatterning for constructing 3D active electrodes.

As shown in the embodiments of FIG. 3B, a microfluidic chip design 330 illustrates a top view of an assembled device. In various aspects, the microfluidic chip design 330 includes the microwell array 380 positioned in a central region, with serpentine microfluidic channels extending from both sides of the microwell array 380. The serpentine channels provide fluid inlet and outlet pathways that enable continuous fluid flow across cell clusters contained within the microwell array 380.

In various embodiments, the microfluidic chip design 330 comprises a microfluidic circuit in fluid communication with the plurality of microwells. As used herein, a “microfluidic circuit” refers to an interconnected network of microchannels, flow passages, manifolds, reservoirs, and/or fluid routing structures configured to deliver, distribute, recirculate, or remove fluids relative to the microwells. The microfluidic circuit may include one or more inlet ports, outlet ports, serpentine channels, distribution manifolds, perfusion chambers, and flow control regions arranged to provide controlled fluid communication with each microwell of the array. The microfluidic circuit may operate under static, perfused, recirculating, or dynamically controlled flow conditions. Fluid communication may include direct channel connection to each microwell, indirect communication through a shared chamber region, or selective addressing of subsets of microwells via controlled flow pathways.

In various embodiments, the microwell array 380 in a fluidic chamber may recapitulate a native physiological niche in which clusters may be protected from shear forces, while still providing an active flow to support long-term culture. The design of the microfluidic chip design 330 creates a continuous fluid flow across the cell clusters in the microwells and delivers a continuous supply of fresh medium rich in oxygen and nutrients and clearance of metabolic wastes by flow. In some aspects, a height of the biochip may be optimized by flow simulation to adjust a thickness of SU-8 mold at various spots across the chip. The microfluidic chip height may be optimized by flow simulation to adjust the thickness of SU-8 mold at various spots across the chip.

In some aspects, by adjusting a magnitude and frequency of an electrical field and controlling a flow rate, cells can be induced to aggregate and form spheroids uniformly. The cells may not attach to a surface of the microwells, which facilitates spontaneous spheroid formation in the absence of a non-adherent coating and allows cells to aggregate and form spheroids through sedimentation and accumulation within the microwells. Injected cells may be trapped within the microwells using a combination of controlled flow and applied electric field, while untrapped cells may be washed out after repeated tilting.

The fabrication process may enable integration of transparent electrodes for optical monitoring compatibility. The integration of transparent ITO electrodes may enable simultaneous optical imaging and electrical monitoring, a capability that may allow for real-time data calibration and validation of viability assays. The development of the microwell array 380 and a multi-well plate chip for generating uniformly sized spheroids through electrical excitation may provide advantages including higher throughput, precise size control, improved reproducibility, cell viability, and compatibility with various cell lines. This label-free and contactless patterning may promote the formation of spheroids of desired sizes, and the system may prevent adhesion of spheroids to channel surfaces, eliminating the need for additional surface treatments.

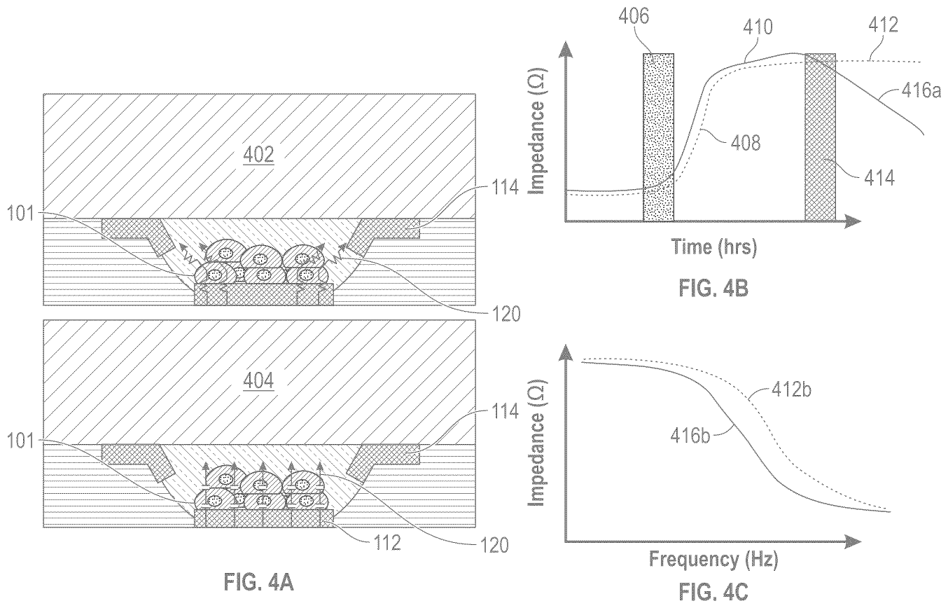

Referring to FIGS. 4A-4C, effects of frequency change on impedance response of microwells using the microwell electrodes are illustrated in accordance with various embodiments. In various aspects, FIGS. 4A-4C depict cross-sectional views of microwells containing the sample 101 along with corresponding impedance response graphs illustrating the effects of frequency and time on impedance measurements.

In the various embodiments shown in a left portion of FIGS. 4A-4C, two cross-sectional views show the microwell configuration with the spiral electrode structure 112 positioned at a bottom surface and the top electrode 114 positioned adjacent to an opening of the microwell 102. For example, the electric field distribution 120 is shown extending between the electrodes and through the sample 101, where an upper cross-section labeled as a low frequency 402 illustrates the electric field distribution 120 at lower frequencies, while a lower cross-section labeled as a high frequency 404 illustrates the electric field distribution 120 at higher frequencies. The electric field distribution 120 may vary based on the frequency of an applied signal, with different frequency ranges providing different characterization capabilities for the sample 101 or 3D spheroid 110 contained within the microwell 102.

With continued reference to the various embodiments shown in FIGS. 4A-4C, a center portion shows a graph plotting impedance in ohms against time in hours, depicting dynamics of real-time impedance changes during spheroid development. In the illustrated embodiments, curves labeled with reference numeral 412 correspond to control groups, while curves labeled with reference numeral 416 correspond to treatment groups. The suffix “a” designates time-domain data (FIG. 4B), and the suffix “b” designates frequency-domain data (FIG. 4C). An impedance curve 412 shows distinct phases including a cells seeded 406 phase at an initial time point, followed by a growth phase 408 where impedance increases, reaching a confluence 410 at a plateau region. Following the confluence 410, a treatment 414 phase is shown where the impedance curve 412 diverges into a treatment group curve 416a showing decreased impedance and a control group curve 412a maintaining higher impedance levels. The divergence between the treatment group curve 416a and the control group curve 412a may indicate differential cellular responses to applied treatments.

As further shown in the embodiments of FIGS. 4A-4C, a right portion shows a graph plotting impedance in ohms against frequency in hertz, displaying representative electrochemical impedance spectroscopy (EIS) curves. A treatment group curve 416b shows a lower impedance response across a frequency range compared to a control group curve 412b, which maintains higher impedance values throughout the frequency spectrum. The comparison between the treatment group curve 416b and the control group curve 412b may provide insights into changes in cellular properties following treatment application.

In various aspects, real-time impedance monitoring and EIS circuit fitting can provide non-invasive insights into assessment of cell behavior, such as changes in cell viability, morphology, and proliferation. In some aspects, the system can perform differential pulse voltammetry (DPV) measurements in addition to EIS and real-time multi-frequency impedance recordings over multiple days in a standard incubator environment. In some cases, the system may perform measurements related to multi-frequency impedance, EIS spectroscopy, and I-V polarization simultaneously, enabling comprehensive characterization of the electrical properties of the spheroids and their heterogeneity.

In some embodiments, voltage ranges of 1-5 mV may be used for planar 2D readings in the 2D mode 104, while voltage ranges of 5-50 mV may be used for 3D out-of-plane measurements in the 3D mode 106, and EIS frequencies may range from 10 Hz to 1 MHz for comprehensive circuit fitting analysis. The multi-frequency monitoring approach may enable modulation of the impedance response from microwells to monitor spheroid heterogeneity. EIS circuit fitting may be employed to quantify different elements of an equivalent circuit model and find an optimal model for characterization of spheroids embedded in the microwells.

In some aspects, the frequency-dependent impedance response may enable differentiation between cell-to-cell interactions and cell-to-media interactions within the 3D spheroid 110 or the sample 101. At the low frequency 402, the impedance measurements may be more sensitive to changes in cell membrane properties and tight junction integrity. At the high frequency 404, the impedance measurements can be more sensitive to changes in cytoplasmic conductivity and overall tissue density. The combination of measurements at the low frequency 402 and the high frequency 404 can advantageously provide comprehensive characterization of both surface dynamics and bulk cellular properties within the three-dimensional structure.

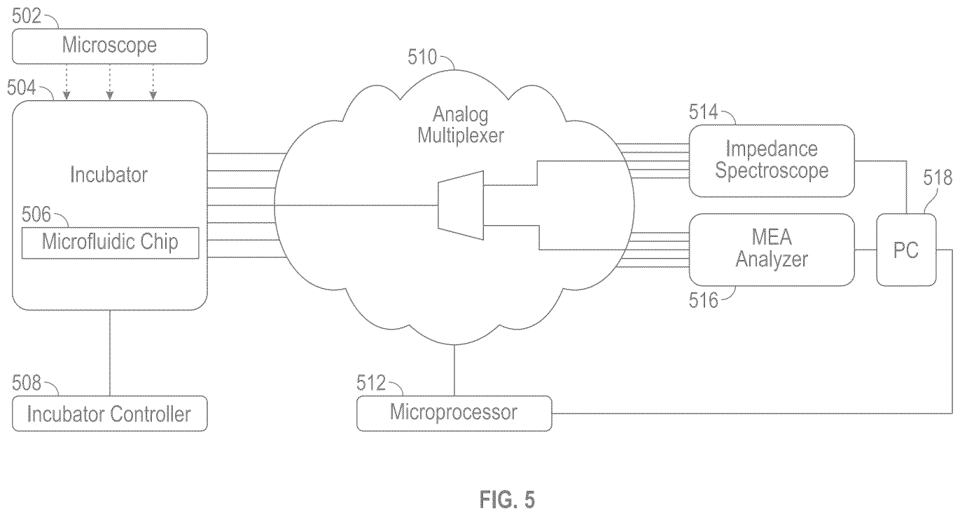

Referring to FIG. 5, an overview of an exemplary multi-modal sensing system 500 for monitoring and characterizing 3D cell cultures is illustrated in accordance with various embodiments. In some embodiments, the multi-modal sensing system 500 may include a microscope 502, an incubator 504, a microfluidic chip 506, an incubator controller 508, an analog multiplexer 510, a microprocessor 512, an impedance spectroscope 514, a microelectrode array (MEA) analyzer 516, and a personal computer (PC) 518.

The microfluidic chip 506 may be positioned within the incubator 504, which maintains controlled environmental conditions for cell culture including temperature, humidity, and gas composition. The incubator controller 508 may be connected to the incubator 504 and may manage operational parameters of the incubator 504 to maintain conditions suitable for long-term culture of the 3D spheroid 110 or the sample 101 contained within the microwell array 380 disposed on the microfluidic chip 506. The microscope 502 may be positioned adjacent to the incubator 504 and may be configured to provide visual and image monitoring of the microfluidic chip 506. The microscope 502 may enable time-lapse imaging of samples contained within the microwell array 380, allowing optical characterization to be performed sequentially or concurrently with electrical sensing. The time-lapse imaging capability may facilitate benchmarking and calibrating spheroid viability assays alongside optical monitoring, providing a holistic understanding of spheroid viability through complementary data from optical and electrical characterization.

The analog multiplexer 510 may be connected to the microfluidic chip 506 and may provide electrical input to and receive output signals from electrode integrated microwells disposed on the microfluidic chip 506. The analog multiplexer 510 may include a printed circuit board (PCB) switchboard that multiplexes analog input and output signals to various microwells on the microfluidic chip 506. The analog multiplexer 510 may enable automatic switching between different microwells or between different sensing modes, such as switching from the 2D mode 104 to the 3D mode 106, or from a single microwell viability mode to an electrophysiological recording mode.

The microprocessor 512 may be connected to the analog multiplexer 510 and may be configured to generate control signals for the analog multiplexer 510. The microprocessor 512 may generate control signals for switching from one microwell to another or from single microwell viability mode to electrophysiological recording. The microprocessor 512 may coordinate operations across the multi-modal sensing system 500, managing the timing and sequencing of measurements from multiple microwells within the microwell array 380. In various embodiments, electrode selection and measurement mode switching may be implemented using analog or digital switching circuitry, multiplexers, programmable logic devices, application-specific integrated circuits (ASICs), field-programmable gate arrays (FPGAs), software-controlled instrumentation interfaces, or combinations thereof. The control architecture may be integrated on-board or distributed across external instrumentation, provided that selective addressing of electrode pairs for planar and through-depth measurements is enabled.

The impedance spectroscope 514 and the MEA analyzer 516 may be connected to the analog multiplexer 510 and may receive signals from the microfluidic chip 506 via the analog multiplexer 510. The impedance spectroscope 514 may perform impedance measurements across a range of frequencies for characterization of electrical properties of samples within the microwells. The MEA analyzer 516 may capture electrophysiological recordings from the samples, enabling monitoring of cellular activity such as action potential propagation and ionic fluxes in electrogenic cells. The multi-modal sensing system 500 may include a lock-in amplifier to minimize noise during multi-frequency impedance measurements. The lock-in amplifier may record data at multiple frequencies, enabling comprehensive characterization of the electrical properties of the 3D spheroid 110 or the sample 101 and their heterogeneity while reducing signal noise.

With continued reference to the embodiments shown in FIG. 5, the personal computer (PC) 518 may be connected to the impedance spectroscope 514 and the MEA analyzer 516 and may receive data from these components for further analysis, storage, and display. The PC 518 may execute machine learning algorithms for data analysis and predictive modeling of cellular behavior based on collected electrical measurements.

In various aspects, machine learning techniques such as Principal Component Analysis (PCA) are applied to impedimetric parameters for extracting neuronal activity peaks during different stages of spheroid growth or disease progression. The PCA can be applied to voltammetric and impedimetric parameters extracted from the 3D spheroid 110 at various stages of development. First principal components obtained from the PCA may serve as inputs for Support Vector Regression (SVR) models, which may be used for predicting spheroid activity. The SVR models can undergo iterative optimization processes to achieve regression results, with each iteration combining random initialization sums and function types along with tuning kernel function parameters and error penalty factors.

The integration of the multi-modal sensing system 500 with machine learning capabilities may facilitate training of predictive models for more accurate and reliable analysis of cellular behavior and drug responses. The multiplexer switch may enable sequential handling of spheroids, generating comprehensive datasets for machine learning training that span various spheroid viability modes. The combination of electrical sensing data from the impedance spectroscope 514 and the MEA analyzer 516 with optical data from the microscope 502 may provide complementary information for self-calibration of the viability assay and validation of measurement accuracy.

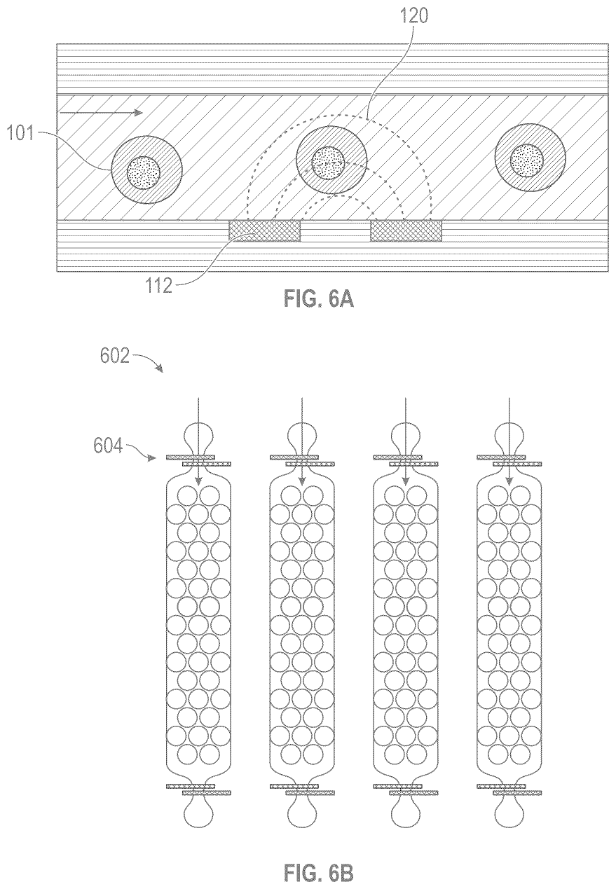

Referring to FIGS. 6A and 6B, cells flowing through a microfluidic impedance cytometer and a multiplexed device integrated with cytometers 602 for precise control over spheroid formation in microwell arrays are illustrated in accordance with various embodiments. FIG. 6A illustrates a cross-sectional view of a microfluidic channel configuration showing the electric field distribution 120 within the channel. The view depicts the sample 101 positioned within the channel structure, with the sample 101 shown as circular cross-sections containing internal cellular structures. The spiral electrode structure 112 may be positioned at a bottom of the channel, indicated by a cross-hatched pattern. The electric field distribution 120 is represented by diagonal hatching throughout the channel area, demonstrating how the electric field extends through the channel space surrounding the sample 101 samples within the channel. For example, dashed lines indicate the interaction between the electric field distribution 120 and the sample 101 samples within the channel.

With continued reference to FIG. 6B, a schematic view of a multiplexed device integrated with the cytometers 602 for precise control over spheroid formation in the microwell array 380 is shown. The configuration shows four parallel vertical channels, each containing electrodes 604 positioned at a top of each channel. The electrodes 604 are depicted with a cross-hatched pattern indicating their conductive nature. Each channel contains multiple circular elements representing cells or spheroids arranged in a vertical column formation. Arrows indicate a direction of flow through each channel, with droplets shown at a bottom of each channel representing output. This arrangement may enable parallel processing of multiple samples through the cytometers 602, facilitating high-throughput analysis and precise control over spheroid formation within the microwell array 380.

In various aspects, impedance flow cytometry may be used as a label-free and non-invasive method for characterizing electrical properties of cells and their heterogeneity. When an alternating current (AC) voltage is applied to a set of cytometer electrodes, a flowing cell perturbs an electrical signal, enabling not only cell detection but also analysis of cell viability without staining. This approach can allow for downstream molecular analysis of the samples.

In some embodiments, the platform may be used for impedance flow cytometry for self-calibration by determining the percentage of live and dead cells in a loaded sample before treatment. In addition to precise cell counting, impedance cytometry may be used to determine the percentage of live and dead cells in the loaded sample before treatment, facilitated by an in-line impedance cytometer. This capability may advantageously ensure improved or accurate control of spheroid dimensions and shapes with higher throughput, promoting consistency and reproducibility across experiments.

As further shown by the various embodiments in FIG. 6B, the multiplexed device with the cytometers 602 may enable scalable parallelization of fluid handling at different treatments. For example, in-line cytometers may be applied to monitor multiplexed cultures and both amplitude and phase change may be used as features for a support vector machine (SVM) classifier to simultaneously assess viability response of loaded cells at different test conditions. These measurements may be executed with a lock-in amplifier to minimize noise, recording data at multiple frequencies. This approach can advantageously establish reproducible conditions for 3D cultures scaled for higher throughput applications.

In various aspects, the platform uses electrical stimulation combining electrophoretic and dielectrophoretic forces to facilitate formation of uniformly sized and shaped spheroids. By leveraging features obtained through cytometry, a quantity of input cells required to generate spheroids of different sizes may be precisely determined. This real-time resource optimization, coupled with multiplexed microwell device monitoring, may enhance economic viability of the platform for various disease modeling applications.

In some embodiments, for data validation, multi-frequency impedance data of cell viability can be cross-referenced with Trypan Blue staining and flow cytometry as ground truth measurements. Following each treatment, spheroids may be dissociated into individual cells using Trypsin-EDTA for post-treatment in-line cytometry. For example, the chip may be tilted repetitively to ensure efficient spheroid dissociation before post-treatment impedance cytometry. The microwell electrode array may be utilized for formation and multiplexed screening of spheroids in dynamic flow conditions, with the electrodes 604 being multimodal and repurposed to stimulate and capture different signals from individual spheroids or multiple interconnected spheroids following their maturation.

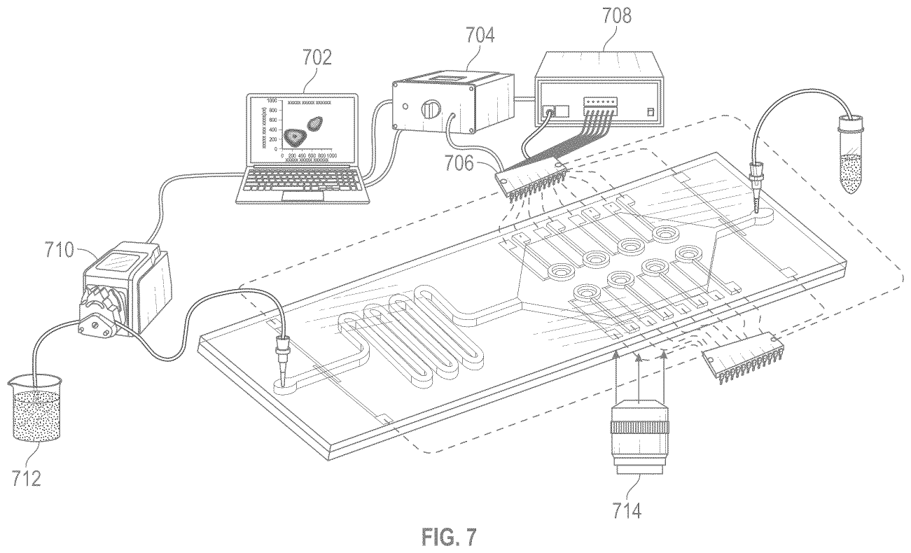

Referring to FIG. 7, FIG. 7 shows various embodiments of an exemplary automated microfluidic platform for label-free quantitative assessment of spheroid viability combined with impedance cytometry and electrophysiological recordings. In various aspects, the automated microfluidic platform includes a machine learning and data analytics module 702 configured to process and analyze data collected from the platform. The machine learning and data analytics module 702 can be connected to a MEA controller 704, which manages operation of the microelectrode array and coordinates signal acquisition from the microfluidic chip 506.

With continued reference to the embodiments shown in FIG. 7, a multichannel connector 706 can provide electrical connections between the MEA controller 704 and the microfluidic chip 506, enabling multiplexed data collection from multiple sample wells within the microwell array 380. The multichannel connector 706 can facilitate simultaneous acquisition of signals from multiple microwells, supporting high-throughput analysis of the 3D spheroid 110 or the sample 101 contained within the microwell array 380.

An impedance analyzer 708 can be coupled to the automated microfluidic platform to perform electrochemical impedance spectroscopy measurements and real-time impedance monitoring of 3D cell cultures within the microwell array 380. In some embodiments, the impedance analyzer 708 may perform measurements across a range of frequencies, enabling comprehensive characterization of electrical properties of samples in both the 2D mode 104 and the 3D mode 106. The impedance analyzer 708 may be configured to measure trans-endothelial electrical resistance (TEER) without the necessity of integrating a conventional TEER setup, enabling non-invasive monitoring of barrier function in the sample 101. Such measurements may be analogous to TEER measurements used in monolayer systems but adapted for three-dimensional tissue constructs.

A peristaltic pump 710 can control fluid flow through the microfluidic channels, delivering fresh medium and removing metabolic waste from the cell cultures contained within the microwell array 380. In various aspects, the peristaltic pump 710 can enable continuous perfusion of the microwell array 380, providing a continuous supply of fresh medium rich in oxygen and nutrients while facilitating clearance of metabolic wastes. A waste container 712 may collect effluent from the microfluidic system, receiving spent medium and cellular debris removed by the fluid flow controlled by the peristaltic pump 710.

In various embodiments, the automated microfluidic platform subjects samples to variable fluid shear stress (FSS) to simulate reperfusion injury post organ transplant. By controlling the peristaltic pump 710, the platform can apply different levels of FSS to the sample 101 or the 3D spheroid 110 contained within the microwell array 380, enabling study of cellular responses to mechanical perturbations. The variable FSS capability may provide a physiologically relevant in vitro model for recapitulating ischemia-reperfusion injury and assisting in optimization of organ preservation protocols.

With continued reference to the embodiments shown in FIG. 7, a live imaging capability 714 can enable optical monitoring of the spheroids concurrently with electrical measurements, facilitating real-time data calibration and validation of the viability assay. The live imaging capability 714 may provide visual confirmation of spheroid morphology and size changes, complementing the electrical data obtained from the impedance analyzer 708 and the MEA controller 704. The integration of the live imaging capability 714 with electrical sensing may provide a holistic understanding of spheroid viability through complementary data from optical and electrical characterization simultaneously.

The machine learning and data analytics module 702 can apply reinforcement learning techniques to continuously optimize the detection process and adapt to real-time data streams. The reinforcement learning techniques may enable the automated microfluidic platform to adjust measurement parameters based on observed cellular responses, improving accuracy and efficiency of the viability assessment over time. The machine learning and data analytics module 702 may process data from the impedance analyzer 708, the MEA controller 704, and the live imaging capability 714 to generate comprehensive analyses of spheroid behavior and drug responses.

In various aspects, the automated microfluidic platform can integrate in-line glucose biosensors for continuous monitoring of glucose uptake and release in recirculating microphysiological systems. The in-line glucose biosensors may provide real-time monitoring of cellular metabolic activity, offering dynamic insights into the microenvironment and enabling precise control of conditions within the microwell array 380. The platform can include fibrous carbon as an immobilization matrix for biorecognition elements to maximize sensor active area of the in-line glucose biosensors.

In some embodiments, a permselective film may be integrated rather than a drop-coated membrane to reduce or minimize sensor-to-sensor variations in the in-line glucose biosensors. The integration of the permselective film can standardize the microfluidic sensor assembly, reducing variability between different sensors and enabling consistent measurements across multiple experiments. The permselective film can enable the in-line glucose biosensors to operate reliably in dynamic flow conditions controlled by the peristaltic pump 710.

The multimodal monitoring approach combining electrical sensing with optical characterization may provide comprehensive analysis of spheroid behavior and drug responses. The combination of impedance measurements from the impedance analyzer 708, electrophysiological recordings from the MEA controller 704, and optical data from the live imaging capability 714 may enable simultaneous assessment of structural and functional properties of the 3D spheroid 110 or the sample 101. The multimodal data acquisition may allow in situ calibration, providing a framework for standardization of complex 3D cell cultures and reducing variability introduced by off-plate measurements and human error.

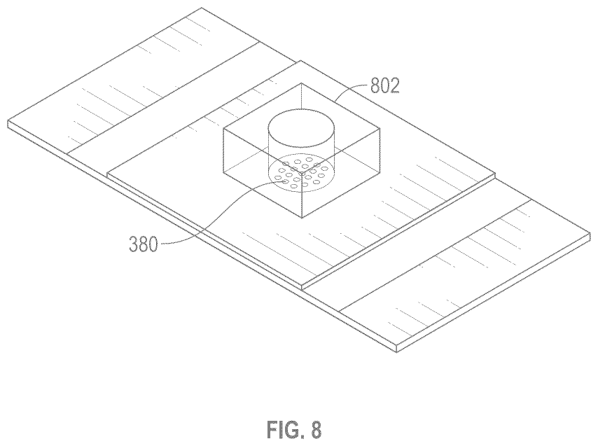

Referring to FIG. 8, an isometric view of the microwell array 380 with a fluidic containment block 802 is illustrated in accordance with various embodiments. In some aspects, the fluidic containment block 802 can be disposed centrally on the microwell array 380 and may feature a cylindrical structure with multiple openings or perforations visible on a surface of the fluidic containment block 802. The fluidic containment block 802 may be formed of polydimethylsiloxane (PDMS) or a similar polymer material, providing biocompatibility and flexibility for interfacing with the microwell array 380.

With continued reference to the embodiments shown in FIG. 8, the fluidic containment block 802 may be configured to interface with the microwell array 380 to facilitate fluid handling and sample processing. The multiple openings in the fluidic containment block 802 may provide fluid inlet and outlet pathways that enable delivery of fresh medium, reagents, and cell suspensions to the microwell array 380, as well as removal of spent medium and metabolic wastes from the cell cultures contained within the microwells.

The microwell array 380 can extend outward from beneath the fluidic containment block 802 in multiple directions, providing a surface area that supports the arrangement of sample wells. Visible line markings on the platform structure of the microwell array 380 may indicate underlying electrode patterns or well boundaries. Electrodes may be integrated into the microwells on top and bottom surfaces of the microwell array 380, enabling both the 2D mode 104 and the 3D mode 106 of impedance measurements. In some cases, electrodes may be integrated along a vertical depth of the microwell array 380, providing additional measurement capabilities for characterizing the 3D spheroid 110 or the sample 101 contained within the microwells.

As further shown in FIG. 8, the configuration of the microwell array 380 with the fluidic containment block 802 may enable integration with microfluidic circuitry for multiplexed screening of cell cultures. The fluidic containment block 802 may provide sealed fluid pathways that connect to external pumping systems, such as the peristaltic pump 710, enabling controlled delivery of fluids to the microwell array 380. The integration of the fluidic containment block 802 with the microwell array 380 may support both two-dimensional and three-dimensional impedance measurements through the electrode arrangements incorporated within the sample wells.

The microfluidic platform incorporating the microwell array 380 and the fluidic containment block 802 may be combined with a tilting mechanism for prevention of cell adhesion to channels and more uniform chip loading and spheroid perfusion. The tilting mechanism may repeatedly tilt the microfluidic platform to prevent cells from adhering to channel surfaces, eliminating the need for additional surface treatments. The repeated tilting may also lead to more uniform chip loading and spheroid perfusion, reducing variability between different microwells within the microwell array 380.

The configuration of the microwell array 380 with the fluidic containment block 802 may provide advantages for high-throughput applications. The multiplexed arrangement of microwells may enable parallel processing of multiple samples, facilitating screening of drug candidates or cellular responses across numerous conditions simultaneously. The integration of electrodes within the microwells may enable real-time monitoring of the 3D spheroid 110 or the sample 101 without interrupting experiments for off-plate measurements.

With continued reference to FIG. 8, the microwell array 380 with the fluidic containment block 802 may be compatible with standard laboratory equipment, including incubators and microscopes. The platform may be positioned within the incubator 504 for maintaining controlled environmental conditions during long-term culture experiments. The transparent electrodes integrated within the microwell array 380 may enable optical monitoring using the microscope 502 concurrently with electrical measurements, providing complementary data for comprehensive analysis of cellular behavior. The compatibility with standard laboratory equipment may reduce the need for specialized instrumentation, enhancing accessibility of the platform for various research applications.

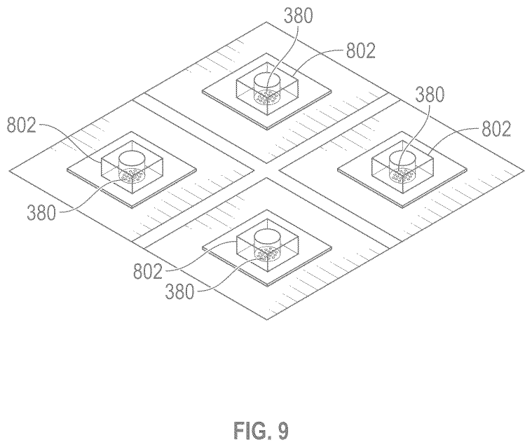

Referring to FIG. 9, an isometric view of a multi-well platform configuration is illustrated in accordance with various embodiments. The platform includes a substrate supporting a plurality of fluidic containment blocks 802 arranged in a symmetric layout. In the illustrated embodiment, four fluidic containment blocks 802 are positioned in a quadrant configuration on a common platform. Each fluidic containment block 802 contains a microwell array 380 configured to accommodate one or more samples 101 for culture and monitoring.

Each microwell array 380 within the respective fluidic containment block 802 may include one or more individual microwell structures configured to support three-dimensional cell culture formation, such as spheroids, organoids, microphysiological systems (MPS), or other biological samples. The fluidic containment blocks 802 may be structurally defined regions configured to retain culture medium and to isolate samples from neighboring blocks, thereby forming distinct compartments for parallel experimentation. The symmetric spatial arrangement of the fluidic containment blocks 802 on the platform may facilitate uniform environmental exposure, consistent electrical routing, and reproducible measurement conditions across multiple sample sites.

In various embodiments, the microwell arrays 380 contained within each fluidic containment block 802 may be electrically addressable individually or in groups, enabling simultaneous or sequential impedance measurements across multiple wells. The platform configuration shown in FIG. 9 may support both planar (2D) impedance measurements and through-depth (3D) impedance measurements, as described elsewhere herein, allowing volumetric and surface-sensitive characterization of multiple samples in parallel. The array-level architecture may further enable multiplexed acquisition of impedance data from different microwell arrays 380 without physical repositioning of the platform.

The distributed arrangement of the fluidic containment blocks 802 and microwell arrays 380 may also facilitate multimodal monitoring. In some embodiments, electrode elements integrated within the microwell arrays 380 may be configured for impedance sensing, electrical stimulation, electrophysiological recording, or combinations thereof. The platform may therefore enable correlation of structural impedance measurements (e.g., cell density, adhesion, barrier integrity, or tissue organization) with functional electrical activity (e.g., action potential propagation, ionic signaling, or other bioelectrical responses) across multiple independent sample regions.

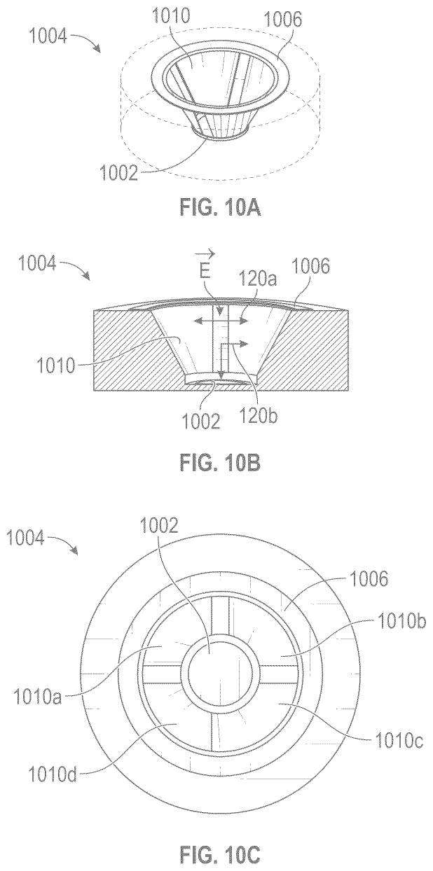

The platform surface may further include alignment features, reference markings, or scale indicators positioned along one or more edges to facilitate reproducible positioning, optical imaging alignment, robotic handling, or integration with external instrumentation. The quadrant arrangement of fluidic containment blocks 802 may allow parallel experimental conditions, comparative analysis between treatment groups, or high-throughput screening applications within a single integrated microwell array platform. Referring to FIGS. 10A-10C, alternative electrode configurations for the microwell array 380 are illustrated for generating electric field distributions within an individual microwell structure 1004 and for enabling three-dimensional (3D) imagery by impedance tomography of a microtissue or other sample 101 disposed within the microwell. FIG. 10A provides an isometric view of an individual microwell structure 1004 within the microwell array 380. The microwell structure 1004 includes sidewalls extending from a wider opening at a top region to a narrower bottom surface, thereby defining a cavity configured to receive and retain the sample 101. A bottom electrode 1002 is disposed at or integrated into the bottom surface of the microwell structure 1004. A top electrode 1006 is positioned proximate to the opening of the microwell structure 1004 and may be implemented as an annular, ring-shaped, or otherwise perimeter-disposed electrode region surrounding the opening. As used herein with respect to FIGS. 10A-10C, element 1004 refers to an individual microwell structure within the microwell array 380, and the sidewalls of the microwell structure 1004 define the cavity in which the sample 101 is contained during culture and monitoring.

FIG. 10B illustrates a cross-sectional side view of the microwell structure 1004 showing the relative positioning of the bottom electrode 1002, the top electrode 1006, and electrode elements disposed along the sidewalls (e.g., a sidewall electrode assembly 1010). In various embodiments, an alternating electrical excitation may be applied between selected electrode elements to establish an electric field \vec{E} within the cavity of the microwell structure 1004 and through the sample 101. When the electrical excitation is applied between the bottom electrode 1002 and the top electrode 1006, a substantially vertically oriented field may be produced such that the electric field \vec{E} has a dominant through-depth component across a depth of the microwell. In such embodiments, an impedance measurement obtained between the bottom electrode 1002 and the top electrode 1006 may be referred to as a through-depth impedance measurement because the current path extends through a bulk of the sample 101 along a depth direction between the bottom region and the top region of the microwell. This through-depth measurement may be particularly sensitive to volumetric or bulk properties of the sample 101, including but not limited to overall tissue conductivity, cell density distribution, internal structural organization, necrotic core development, or other depth-dependent heterogeneity.

As further shown in FIG. 10B, the electric field distribution within the microwell structure 1004 may include multiple components that can be selectively emphasized depending on which electrode elements are activated. In some embodiments, a first electric field component 120a corresponds to a vertically oriented field component (e.g., a dominant axial component) established between the bottom electrode 1002 and the top electrode 1006 to enable through-depth impedance measurements across the cavity. In addition to the vertically oriented field component 120a, lateral, radial, transverse, and/or circumferential electric field components may be generated within the cavity depending on which electrode elements are activated. The vector E illustrated in FIG. 10B (e.g., electric field \vec{E}) schematically represents the resultant electric field established within the cavity, which may comprise a dominant axial component (120a), one or more lateral or radial components (120b), or a composite field resulting from simultaneous activation of multiple electrode elements. For example, a second electric field component 120b may correspond to a lateral or radial field component established between the bottom electrode 1002 and one or more electrode segments disposed along the sidewalls (e.g., one or more portions of the sidewall electrode assembly 1010), or between different sidewall electrode segments, thereby producing an electric field \vec{E} having a substantial horizontal, radial, circumferential, or multi-directional component within the cavity. In various embodiments, the vertical field component 120a and the lateral/radial/circumferential field component 120b may be applied independently, sequentially, or in combination to obtain impedance data from different spatial orientations relative to the sample 101.

FIG. 10C shows a top view of the microwell structure 1004. In this embodiment, a sidewall electrode assembly 1010 is disposed along interior surfaces of the sidewalls of the microwell structure 1004. The sidewall electrode assembly 1010 may be circumferentially distributed and segmented into a plurality of discrete electrode regions that are electrically isolated and independently addressable, such as segmented regions 1010a, 1010b, 1010c, and 1010d arranged around the cavity perimeter. The top electrode 1006 may be implemented as a ring-shaped electrode surrounding the opening, and the bottom electrode 1002 may be positioned centrally at the bottom surface of the cavity. By selectively activating one or more of the segmented sidewall electrode regions 1010a-1010d, different angular and/or radial field orientations may be generated within the microwell cavity, including fields directed toward a particular quadrant or circumferential region of the sample 101.

In various embodiments, impedance measurements may be obtained using different measurement topologies to provide complementary spatial sensitivity. For example, a first measurement topology may include applying an excitation signal between the bottom electrode 1002 and the top electrode 1006 and measuring a corresponding response to obtain through-depth impedance data (e.g., corresponding to the vertical field component 120a). A second measurement topology may include applying an excitation signal between the bottom electrode 1002 and one selected sidewall electrode segment (e.g., 1010a) and measuring a corresponding response at another sidewall electrode segment (e.g., 1010c), or measuring between the selected sidewall electrode segment and the bottom electrode 1002, to obtain lateral, radial, or circumferential impedance data (e.g., corresponding to the field component 120b). In further embodiments, sequential switching among sidewall electrode segments 1010a-1010d (e.g., energizing different segments as driving electrodes and using other segments as receiving electrodes) may enable multi-angle field projection and angularly resolved impedance measurements without physical repositioning of the sample 101.

By acquiring impedance measurements under multiple electrode activation patterns and field orientations (including through-depth measurements associated with 120a and lateral/radial/circumferential measurements associated with 120b), the system may reconstruct an impedance distribution of the sample 101 to generate a three-dimensional representation or 3D imagery by impedance tomography. In such embodiments, the reconstructed impedance distribution may provide spatial mapping of electrical properties within the sample 101, including identification of localized inhomogeneities, depth-dependent gradients, anisotropic tissue organization, or viability-related structural changes. In some embodiments, the tomographic reconstruction may be performed using a plurality of impedance measurements obtained by energizing different combinations of electrode elements (e.g., bottom-to-top, bottom-to-sidewall, and sidewall-to-sidewall) and using the resulting measurement set to compute a volumetric or spatially resolved impedance map of the microtissue.

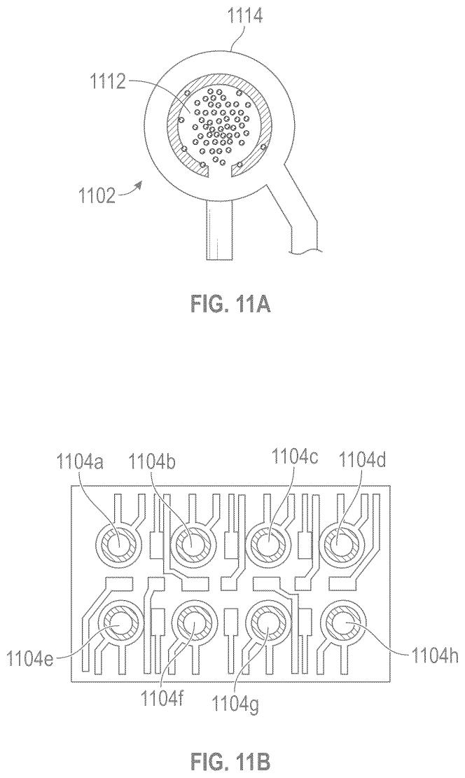

In some embodiments, the bottom electrode 1002 may form part of the first electrode structure and may be positioned along or integrated with a lower substrate of the microwell array 380. In certain embodiments, the top electrode 1006 may be positioned along or integrated with an upper substrate or cover layer and may form part of a second electrode layer. In various embodiments, the sidewall electrode assembly 1010 may be formed as conductive traces disposed on, embedded within, or otherwise integrated along interior surfaces of the sidewalls of the microwell structure 1004. The combination of the bottom electrode 1002, the top electrode 1006, and the sidewall electrode assembly 1010 may provide enhanced spatial resolution for impedance measurements and enable selective generation of the vertical field component 120a and the lateral/radial/circumferential field component 120b for multi-directional sensing and impedance tomography. Referring to FIGS. 11A and 11B, collective and individual microwell monitoring configurations are illustrated in accordance with various embodiments. FIG. 11A illustrates a top view of a collective continuous electrode for the microwell array 1102, which includes a top electrode 1112 and a bottom electrode 1114. In this configuration, a signal may be averaged across a whole device area, with the top electrode 1112 functioning as a top electrode and the bottom electrode 1114 functioning as a bottom electrode. The collective continuous electrode for the microwell array 1102 may enable monitoring of aggregate properties across multiple microwells simultaneously.

With continued reference to FIG. 11B, a photographic image of an arrayed electrode configuration is shown with multiple microwells arranged in a grid pattern. The array includes a first microwell 1104a, a second microwell 1104b, a third microwell 1104c, a fourth microwell 1104d, a fifth microwell 1104e, a sixth microwell 1104f, a seventh microwell 1104g, and an eighth microwell 1104h. Each of the individual electrical connections for each of the first microwell 1104a through the eighth microwell 1104h may be electrically separated so that the system may collect independent signals from specific microwells rather than a single bulk average as compared to the collective continuous electrode for the microwell array 1102 shown in FIG. 11A.

Collective data monitoring using the collective continuous electrode for the microwell array 1102 may provide advantages including simplified data acquisition, reduced complexity of electrical connections, and faster measurement cycles across the entire microwell array 380. The collective monitoring approach may be suitable for applications where aggregate properties of multiple spheroids are of interest, such as screening for overall population responses to treatments.

Individual microwell monitoring using the electrically separated connections to the first microwell 1104a through the eighth microwell 1104h may provide advantages including spheroid-to-spheroid monitoring that captures unique properties and behavior of each spheroid within the array. This approach may be particularly valuable for identifying heterogeneities between spheroids, such as differences in cell density, viability, or drug response, which might be masked in collective data. By enabling high-resolution, localized analysis, single-microwell monitoring may provide a deeper understanding of individual spheroid properties, complementing broader insights obtained from collective data monitoring.

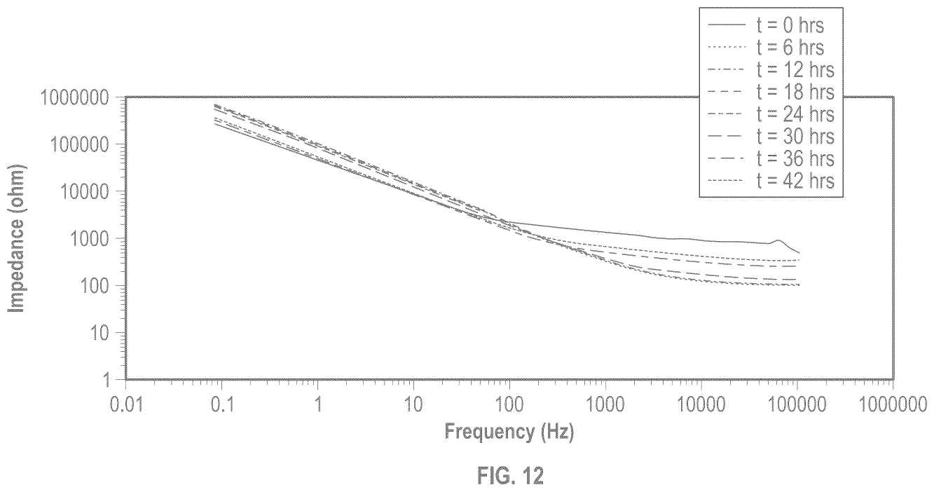

Referring to FIG. 12, an exemplary impedance Bode plot showing electrical properties of the microwell array during spheroid formation over time is illustrated in accordance with various embodiments. The impedance Bode plot displays impedance in ohms on a vertical axis ranging from 1 to 1,000,000 ohms on a logarithmic scale, and frequency in Hertz on a horizontal axis ranging from 0.01 Hz to 1,000,000 Hz on a logarithmic scale. Multiple curves are shown representing measurements taken at different time points during spheroid formation, including measurements at t=0 hrs, t=6 hrs, t=12 hrs, t=18 hrs, t=24 hrs, t=30 hrs, t=36 hrs, and t=42 hrs. Each time point is represented by a distinct line style as indicated in a legend accompanying the plot.

With continued reference to the embodiments shown in FIG. 12, the curves demonstrate a general trend of decreasing impedance as frequency increases across all time points. The gradual transition in impedance reflected in the curves may indicate a balance between cellular adhesion and medium conductivity within the microwell array. As spheroid formation progresses over the 42-hour period following cell seeding, electrode-cell interactions may increase, altering capacitive behavior of the system. The changes in impedance over time may reflect structural and organizational changes occurring within the developing spheroids as cells aggregate and form three-dimensional structures.

At lower frequencies below approximately 10 Hz, the curves show relatively high impedance values and exhibit some separation between different time points. As the spheroid matures, tighter cell-cell junctions may form within the three-dimensional structure, limiting ionic movement through the microtissue. This limitation of ionic movement may contribute to increased impedance observed at low frequencies in later time points compared to earlier time points. The low frequency impedance response may be sensitive to changes in cell membrane properties and tight junction integrity within the developing spheroid.

As frequency increases toward a middle range between approximately 10 Hz and 10,000 Hz, the curves converge and follow a similar downward slope. At higher frequencies above approximately 10,000 Hz, the curves begin to diverge again, with later time points showing higher impedance values compared to earlier time points. When cells are seeded at the initial time point, the cells may be more dispersed, and an electrical signal may primarily pass through the culture medium, which has a relatively low dielectric constant. As the cells begin to aggregate and form spheroids over time, a density of biological material within the microwells may increase, leading to a higher effective permittivity as shown in the impedance Bode plot.

The data captured using electrodes positioned at both bottom and top layers of the microwell array in the 3D assay mode may illustrate changes in electrical properties as spheroids mature within the microwells over the 42-hour period following cell seeding. The 3D assay mode may enable capture of data over a wide frequency range, from 100 millihertz to 100 kilohertz as shown in FIG. 12. In certain embodiments, a frequency range of 100 millihertz to 100 kilohertz may be used, while broader ranges (e.g., 10 Hz to 1 MHz) may be employed in other embodiments for comprehensive circuit fitting analysis. This wide frequency range may provide comprehensive characterization of spheroid development, enabling detection of changes in both surface properties and bulk tissue characteristics as the spheroid forms and matures.

The impedance Bode plot analysis may provide advantages for monitoring spheroid formation in real-time without interrupting experiments for off-plate measurements. The frequency-dependent impedance response captured in the 3D assay mode may enable differentiation between various stages of spheroid development, from initial cell seeding through aggregation and maturation. The combination of low frequency and high frequency measurements may provide complementary information about cellular organization, junction formation, and overall tissue density within the developing spheroid. The 3D assay mode data collection approach may facilitate comprehensive characterization of spheroid development by capturing volumetric impedance measurements throughout the three-dimensional structure of the microwell.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms, even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding,” whether explicitly discussed or not, and, conversely, were there only disclosure of the act of “protruding,” such a disclosure should be understood to encompass disclosure of a “protrusion.” Such changes and alternative terms are to be understood to be explicitly included in the description.

A number of implementations have been described. Nevertheless, it will be understood that various modifications may be made without departing from the spirit and scope of the disclosure. Accordingly, other implementations are within the scope of the following claims.

1. A system for multi-modal monitoring of three-dimensional biological samples, comprising:
a platform comprising a plurality of microwells, each microwell of the plurality of microwells being defined by a sidewall and a bottom surface and configured to receive a sample;
a first electrode structure disposed within each microwell of the plurality of microwells, wherein the first electrode structure comprises a pair of coplanar conductive elements; and
a second electrode disposed adjacent to each microwell of the plurality of microwells,
wherein the pair of coplanar conductive elements are independently controllable to obtain a two-dimensional impedance measurement, and wherein at least a portion of the first electrode structure and the second electrode are controllable to obtain a three-dimensional impedance measurement.

2. The system of claim 1, wherein at least one of the first electrode structure or the second electrode is formed of a transparent conductive material.

3. The system of claim 2, wherein the transparent conductive material comprises indium-tin-oxide.

4. The system of claim 1, further comprising a microfluidic circuit in fluid communication with the plurality of microwells.

5. The system of claim 4, wherein the microfluidic circuit comprises serpentine channels configured to deliver fluid flow across the plurality of microwells.

6. The system of claim 1, wherein the first electrode structure comprises a spiral-shaped coplanar electrode pair.

7. The system of claim 1, wherein a spacing between the first electrode structure and the second electrode is in a range of about 300 to about 500 microns.

8. The system of claim 1, further comprising an analog multiplexer configured to selectively connect to individual microwells of the plurality of microwells.

9. The system of claim 8, further comprising a microprocessor configured to generate control signals for the analog multiplexer to switch between the two-dimensional impedance measurement and the three-dimensional impedance measurement.

10. The system of claim 1, wherein the first electrode structure and the second electrode are configured to apply an electrical field to induce cell aggregation within the plurality of microwells.

11. The system of claim 1, wherein the two-dimensional impedance measurement captures cell-to-media interactions at a surface of a sample disposed within a microwell of the plurality of microwells, and wherein the three-dimensional impedance measurement captures cell-to-cell interactions within a bulk of the sample.

12. A method for monitoring three-dimensional cell cultures, comprising:
providing a platform comprising a plurality of microwells, each microwell comprising a first electrode structure disposed at a surface of the microwell, the first electrode structure comprising a pair of coplanar conductive elements, and a second electrode disposed adjacent to the microwell;
introducing cells into the plurality of microwells;
applying an electrical signal between at least one conductive element of the first electrode structure and the second electrode to obtain a three-dimensional impedance measurement of a cell culture within the microwell; and
monitoring a formation or viability of the cell culture based on the three-dimensional impedance measurement.

13. The method of claim 12, further comprising applying an electrical signal between the pair of coplanar conductive elements of the first electrode structure to obtain a two-dimensional impedance measurement.

14. The method of claim 13, wherein the two-dimensional impedance measurement captures cell-to-media interactions at a surface of the cell culture, and wherein the three-dimensional impedance measurement captures cell-to-cell interactions within a bulk of the cell culture.

15. The method of claim 12, further comprising applying an electrical field between the first electrode structure and the second electrode to induce aggregation of the cells into a spheroid within the microwell.

16. The method of claim 15, wherein applying the electrical field comprises adjusting a magnitude and a frequency of the electrical field to control a size of the spheroid.

17. A microfluidic device for monitoring three-dimensional cell cultures, comprising:
a microwell array comprising a plurality of microwells configured to accommodate spheroid formation therein;
a first electrode layer disposed within the microwell array, the first electrode layer comprising a plurality of electrode structures, each electrode structure comprising a pair of coplanar conductive elements positioned within a corresponding microwell;
a second electrode layer disposed at a top position of the microwell array, the second electrode layer being positioned adjacent to the plurality of microwells; and
a microfluidic channel in fluid communication with the plurality of microwells,
wherein the first electrode layer and the second electrode layer are configured to enable planar impedance measurements within a bottom region of each microwell and through-depth impedance measurements across a depth of each microwell.

18. The microfluidic device of claim 17, wherein at least one of the first electrode layer or the second electrode layer comprises indium-tin-oxide.

19. The microfluidic device of claim 18, wherein both the first electrode layer and the second electrode layer comprise indium-tin-oxide, enabling simultaneous optical imaging and impedance measurements of spheroids within the plurality of microwells.

20. The microfluidic device of claim 17, wherein the first electrode layer comprises spiral-shaped coplanar electrode structures configured to capture planar impedance measurements from a surface of a spheroid disposed within a microwell of the plurality of microwells.
